(19) United States
(12) Patent Application Publication
(10) Pub. No.: US 2018/0168736 A1
(43) Pub. Date: Jun. 21, 2018

(54) SURGICAL NAVIGATION SYSTEM AND INSTRUMENT GUIDING METHOD FOR THE SAME

(71) Applicant: SATURN IMAGING INC., New Taipei City (TW)

(72) Inventor: Been-Der Yang, New Taipei City (TW)

(21) Appl. No.: 15/829,949

(22) Filed: Dec. 3, 2017

(30) Foreign Application Priority Data
Dec. 15, 2016 (TW) 105141589

(51) Int. Cl. A61B 34/20; A61B 5/00; A61B 6/03; A61B 8/08; A61M 35/00

(52) U.S. Cl. CPC A61B 34/20; A61B 5/0066; A61B 6/032; A61B 2090/366; A61M 35/00; A61B 2034/2065; A61B 8/0841

(57) ABSTRACT

The present disclosure provides a surgical navigation system and an instrument guiding method for the same. The instrument guiding method includes: receiving three-dimensional (3D) space information of a predetermined instrument path of an instrument; transmitting the 3D space information to a processing unit such that the processing unit converts the three-dimensional space information to two-dimensional (2D) space information by using a projection model algorithm; and receiving, by at least two image-type projecting units, the 2D space information and projecting at least two patterns into a physical space, wherein the at least two patterns intersect each other to form an intersection area.

### TECHNICAL FIELD

The present disclosure relates to surgical navigation systems and instrument guiding methods for the same, and, more particularly, to a surgical navigation system and an instrument guiding method for the same that increase the convenience of surgical operations by providing optical guidance.

### BACKGROUND

In today's minimally invasive surgeries, surgeons often perform operations based on information from preoperative images or real-time images. Surgical navigation systems aid surgeons in performing operations. Current surgical navigation systems that are commonly seen include, for example, the applications of ultrasound imaging or infrared imaging in conjunction with pre-operative images (such as magnetic resonance images, computed tomography images, X-ray images).

However, in existing surgical navigation systems, regardless of the use of preoperative images or real-time images (e.g., real-time images provided by ultrasound), a surgeon performing an operation has to switch focus between an image screen provided by the surgical navigation system and the physical space of the patient where the operation is being carried out. This is inconvenient and may even lead to errors in the operation.

Therefore, there is a need for a surgical navigation system and an instrument guiding method for the same that address the aforementioned issues in the prior art.

### SUMMARY

In view of the aforementioned shortcomings of the prior art, the present disclosure is to provide a surgical navigation system, which may include: a navigation unit for obtaining three-dimensional (3D) space information of a predetermined operation path of an instrument; a processing unit for receiving the 3D space information and converting the 3D space information into two-dimensional (2D) space information by using a projection model algorithm; and at least two image-type projecting units for receiving the 2D space information respectively and projecting at least two patterns in a physical space, wherein the two patterns intersect each other to form an intersection area.

The present disclosure is also to provide an instrument guiding method for a surgical navigation system, which may include: obtaining, by a navigation unit, three-dimensional (3D) space information of a predetermined operation path of an instrument; transmitting the 3D space information to a processing unit and converting, by the processing unit, the 3D space information into two-dimensional (2D) space information by using a projection model algorithm; and receiving, by at least two image-type projecting units, the 2D space information and projecting at least two patterns in a physical space, wherein the two patterns intersect each other to form an intersection area.

With the surgical navigation system and the instrument guiding method for the same according to the present disclosure, the 3D space information of a predetermined operation path of an instrument can be converted into the 2D space information using a processing unit, such that at least two image-type projecting units can project two patterns in the physical space. The intersection area of the two patterns is the guiding path of the surgical instrument. As such, a surgeon does not need to switch focus between an image screen provided by a traditional surgical navigation system and the physical space of the patient where operation is being performed. The surgeon is able to start operation based on the guiding path of the surgical instrument. This increases convenience in operations of the surgery.

### BRIEF DESCRIPTION OF THE DRAWINGS

The present disclosure can be more fully understood by reading the following detailed description of the embodiments, with reference made to the accompanying drawings, wherein:

FIG. 1 is a schematic diagram depicting arrangement of a surgical navigation system in accordance with a first embodiment of the present disclosure;

FIG. 2 is a schematic diagram depicting arrangement of a surgical navigation system in accordance with a second embodiment of the present disclosure;

FIG. 3 is a schematic diagram depicting arrangement of a surgical navigation system in accordance with a third embodiment of the present disclosure;

FIG. 4 is a schematic diagram depicting application of a surgical navigation system of the present disclosure;

FIG. 5A is a schematic diagram depicting application of a surgical navigation system in accordance with a fourth embodiment of the present disclosure;

FIG. 5B is a schematic diagram depicting application of a surgical navigation system in accordance with a fifth embodiment of the present disclosure;

FIG. 6 is a schematic diagram depicting application of a surgical navigation system in accordance with a sixth embodiment of the present disclosure; and

FIG. 7 is a flowchart depicting an instrument guiding method for a surgical navigation system in accordance with an embodiment of the present disclosure.

### DETAILED DESCRIPTION OF THE EMBODIMENTS

The present disclosure is described by the following specific embodiments. Those with ordinary skills in the arts can readily understand other advantages and functions of the present disclosure after reading the disclosure of this specification. The present disclosure may also be practiced or applied with other different implementations. Based on different contexts and applications, the various details in this specification can be modified and changed without departing from the spirit of the present disclosure.

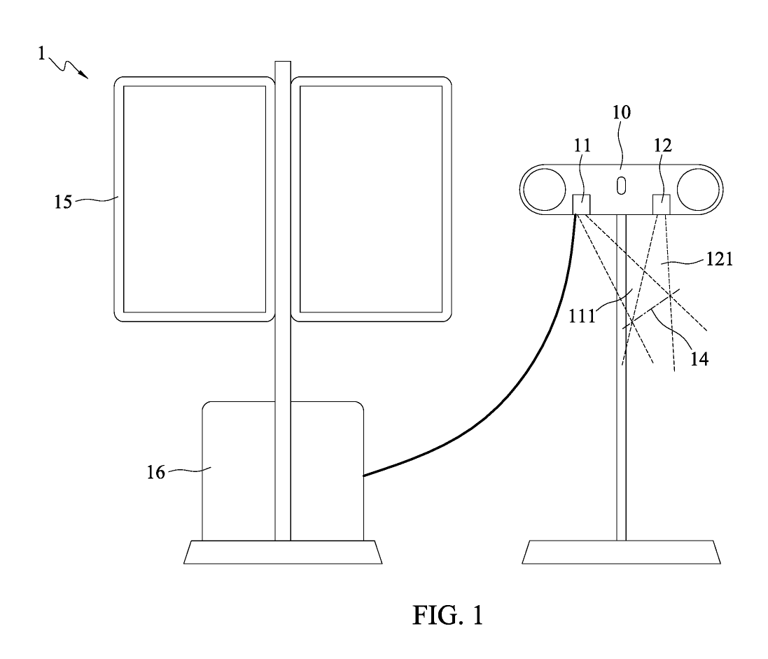

Referring to FIG. 1, a surgical navigation system 1 in accordance with the first embodiment of the present disclosure includes a navigation unit 10, a processing unit 16 and at least two image-type projecting units. The present disclosure does not limit the number of image-type projecting units. A first image-type projecting unit 11 and a second image-type projecting unit 12 are used for illustration purposes only. The first image-type projecting unit 11 and the second image-type projecting unit 12 are used for projecting small matrix images into a space, and can be pico projectors, such as Digital Light Processing (DLP) projecting devices, Laser Beam Scanning (LBS) projecting devices or Liquid Crystal on Silicon (LCoS) projecting devices, but the present disclosure is not limited thereto.

More specifically, the image-type projecting units according to the present disclosure are image-type projecting devices for receiving data, such as video data or/and image data, and projecting patterns into a physical space based on the received video or/and image data. Thus, in an embodiment the image-type projecting devices include a video transmission interface, such as a High Definition Multimedia Interface (HDMI), a Video Graphics Array (VGA) or a DisplayPort.

A preferred embodiment of the present disclosure includes the use of LBS projecting devices, which have the advantage of being focus free, such that a clear intersecting image can be formed in the physical space. Moreover, its raster-scanned single-pixel beam provides images with higher luminance, resulting in human eyes to see brighter images due to visual persistence.

In an embodiment, the first image-type projecting unit 11 and the second image-type projecting unit 12 are installed on the navigation unit 10. As a result, the conversions of the coordinate systems between the first image-type projecting unit 11/second image-type projecting unit 12 and the navigation unit 10 are fixed and known in advance.

The navigation unit 10 is used for obtaining three-dimensional (3D) space information of a predetermined operation path of an instrument. In an embodiment, the 3D space information of a predetermined operation path of an instrument can be obtained by an optical tracker (e.g., an infrared tracker). In other words, the navigation unit 10 can be provided with an infrared tracker. When a reflective ball mark is provided on the instrument, the navigation unit 10 is able to track in real time the current location of the instrument via the infrared tracker. In other embodiments, the 3D space information of a predetermined operation path of an instrument can be obtained by other types of trackers (e.g., a magnetic tracker, a mechanical tracker), ultrasound, computed tomography (CT), magnetic resonance imaging (MRI), optical coherence tomography (OCT) or the like.

More specifically, the 3D space information of a predetermined operation path of an instrument can be obtained before the operation or during the operation in real time. That is, the navigation unit 10 can be categorized as a pre-operative imaging system, an intra-operative imaging system or an intra-operative real-time imaging system. In the pre-operative imaging system in which an infrared tracker is used in conjunction with a pre-operative image (CT image or MRI image), for example, the current actual location of a patient has to be registered with the images obtained by CT or MRI for the image registration process. In the intra-operative imaging system, in which, for example, images are obtained by CT or MRI, there is no need for the image registration process, since the patient is in the CT equipment or MRI equipment during image capturing and operation. The patient stays still after the image is taken, so the actual location of the patient is aligned with the image, meaning that the image registration process is not necessary. In the intra-operative real-time imaging system, in which, for example, images are obtained by ultrasound, there is no need for the image registration process. The various implementations of the image registration process are well-known to those having ordinary skill in the art and thus will not be illustrated further.

The images obtained by CT or MRI can be provided as an pre-operative image, in which case the image registration process is required and performed in conjunction with a tracker, or as an intra-operative image, in which case no image registration process is needed.

In an embodiment, the surgical navigation system 1 provides surgical navigation through the use of the navigation unit 10 (e.g., an infrared tracker) in conjunction with pre-operative images (displayed by a display unit 15). The pre-operative images of a patient can be obtained by CT scanning equipment, MRI scanning equipment or other medical imaging equipment before the operation. There are two different implementation contexts in which the 3D space information of a predetermined operation path of an instrument is obtained. In one implementation context, the surgical navigation system 1 provides a software (e.g., user interface) to allow the surgeon to plan the operation beforehand, for example, deciding on the location and angle of an entry point based on the various image slices of the pre-operative images. During the operation, the infrared tracker (i.e., the navigation unit 10) is used to register the current actual location of the patient with the pre-operative image location, and the location and angle of the planned entry point are obtained (i.e., 3D information of a predetermined operation path is obtained via the software interface), then the processing unit 16 is used for converting the 3D space information into 2D space information by using a projection model algorithm, such that the first image-type projecting unit 11 and the second image-type projecting unit 12 are able to project patterns in the physical space based on the received 2D space information so as to indicate the location and the angle of the entry point for the surgery.

In the other implementation context, the location and the angle of an entry point are decided during the operation. For example, in the case that a tracker is used, after the image registration is performed, the surgeon can hold, for example, a surgical instrument equipped with a tracking ball to allow the navigation unit 10 to track the tracking ball and thus locate the surgical instrument, and the display unit 15 displays a pre-operative image and the current real-time location of the surgical instrument (i.e., the real-time location of the surgical instrument is superimposed on the pre-operative images). This allows the surgeon to view the pre-operative images and the real-time location of the surgical instrument simultaneously to simulate the angle and the location of an entry point of the surgical instrument on the patient. Once the surgeon confirms the angle and the location of an entry point of the surgical instrument, an instruction can be inputted into the navigation unit 10 (e.g., by pressing a confirmation button on the surgical instrument, by operating an input device, such as a mouse, a pedal or a keyboard of the surgical navigation system 1 etc.), and the determined angle and the location of an entry point become the predetermined operation path of the surgical instrument. The navigation unit 10 can then converts the predetermined operation path into 3D space information.

The processing unit 16 is used for receiving the 3D space information and converting the 3D space information into 2D space information using a projection model algorithm. The 2D space information can be video or image data. The image-type projecting units can then receive the 2D space information via a video transmission interface. In an embodiment, the projection model algorithm is a perspective projection model, and has a formula as s\tilde{m}=P\tilde{M}, P=K[R|t], wherein M is the 3D space information of the instrument path under the coordinate system of the navigation unit 10, m is the 2D space information of the instrument path under the coordinate system of the projection model, s is a scaling parameter, and P is a projection matrix that includes K being a projection calibration matrix, R being a rotational matrix, and t being a translation vector. Therefore, m can be obtained from M using this algorithm. In other words, 2D space information for the image-type projecting units can be derived from the 3D space information. Furthermore, in an embodiment, the scaling parameter is usually set to 1, but the present disclosure is not so limited. The present disclosure does not limit the projection model algorithm used.

In an embodiment, conversions of the coordinate systems between the first image-type projecting unit 11/the second image-type projecting unit 12 and the navigation unit 10 are fixed and known in advance, which means that R and t are fixed and known.

After the 2D space information are obtained by the processing unit 16, the first image-type projecting unit 11 and the second image-type projecting unit 12 receive the 2D space information and project each pattern, respectively, in the physical space (i.e., at least two patterns are projected from the two image-type projecting units). For example, the first image-type projecting unit 11 projects a first pattern 111 and the second image-type projecting unit 12 projects a second pattern 121. The first pattern 111 and the second pattern 121 intersect each other to form an intersection area 14. The intersection area 14 provides an indication of the angle and the location for the surgical instrument to be operated on the patient. This aspect will be described in more details later.

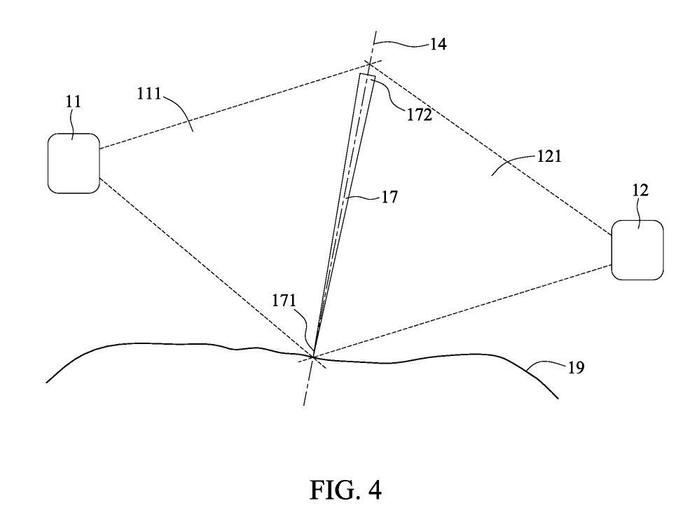

As shown in FIG. 4, the first image-type projecting unit 11 and the second image-type projecting unit 12 project the first pattern 111 and the second pattern 121, respectively. The first pattern 111 and the second pattern 121 form the intersection area 14 in the space above a patient 19 where operation is to be performed, wherein the intersection area 14 can be formed by straight lines or curved lines. The intersection area 14 is shown in straight lines for illustration purposes only. The surgeon then points a first end 171 of a surgical instrument 17 at a point on the patient 19 onto which the intersection area 14 projects, and then rotates a second end 172 of the surgical instrument 17 using the first end 171 as a pivot point until the second end 172 of the surgical instrument 17 overlaps the intersection area 14. Once they overlap, the surgical instrument 17 is at the angle and location ready for operation.

In another embodiment, the surgical navigation system 1 according to the present disclosure further includes a medium spreading unit. The medium spreading unit can be provided as a stand-alone device having a wireless interface for receiving wireless signals. The medium spreading unit receives an instruction from the surgical navigation system 1 and spreads a medium into the physical space based on the instruction to show the intersection area 14 in helping the surgeon identifying the intersection area 14 created by the surgical navigation system 1. The medium can be a material with scattering characteristics (e.g., high-concentration silicon dioxide, titanium dioxide, dry ice or other sterilized materials with high scattering coefficients). The medium spreading unit can be, for example, a sprayer or other spraying devices, and the present disclosure is not so limited.

Moreover, the surgical navigation system 1 according to the present disclosure may include the display unit 15 and the processing unit 16 connected with the navigation unit 10. The display unit 15 is used for displaying pre-operative images or intra-operative real-time images processed by the processing unit 16.

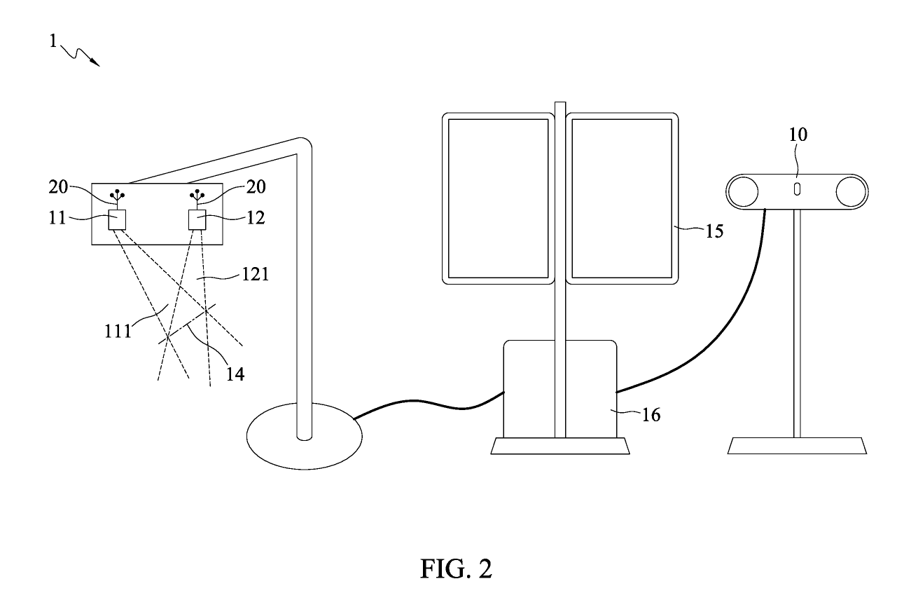

Referring to FIG. 2, a surgical navigation system 1 in accordance with the second embodiment of the present disclosure also includes a navigation unit 10, a first image-type projecting unit 11, a second image-type projecting unit 12 and a processing unit 16. Only the differences between the first and second embodiments are described below, while similar or the same technical contexts are omitted for conciseness.

The first image-type projecting unit 11 and the second image-type projecting unit 12 are not provided on the navigation unit 10, but on another supporting element. Therefore, the relationship of the coordinate systems between the first image-type projecting unit 11 and the second image-type projecting unit 12 is fixed, but the relationship of the coordinate systems between the first image-type projecting unit 11/second image-type projecting unit 12 and the navigation unit 10 is not fixed. In other words, the conversions of the coordinate systems between the image-type projecting units and the navigation unit 10 are not fixed and not known. The locations of the image-type projecting units have to be located (i.e., via tracking balls 20) before coordinate conversion can be performed. In other words, R and t are not fixed and are determined through real-time detecting the locations of the image-type projecting units. In this embodiment, the surgeon can move the supporting element at will to adjust the projection locations of the first image-type projecting unit 11 and the second image-type projecting unit 12.

It should be noted that the image-type projecting units can be located by an optical tracker, an electromagnetic tracker or a mechanical tracker (e.g., a gyroscope and an accelerator) to establish conversions of the coordinate systems between the image-type projecting units and the navigation unit 10. In an embodiment, tracking balls 20 are provided on the image-type projecting units to enable an infrared tracker (i.e., the navigation unit 10) to track the image-type projecting units and establish conversion relationships of the coordinate systems between the image-type projecting units and the navigation unit 10. The infrared tracker and the tracking balls above are merely an embodiment of the present disclosure, and the present disclosure does not limit the types and arrangements of the locating and located devices.

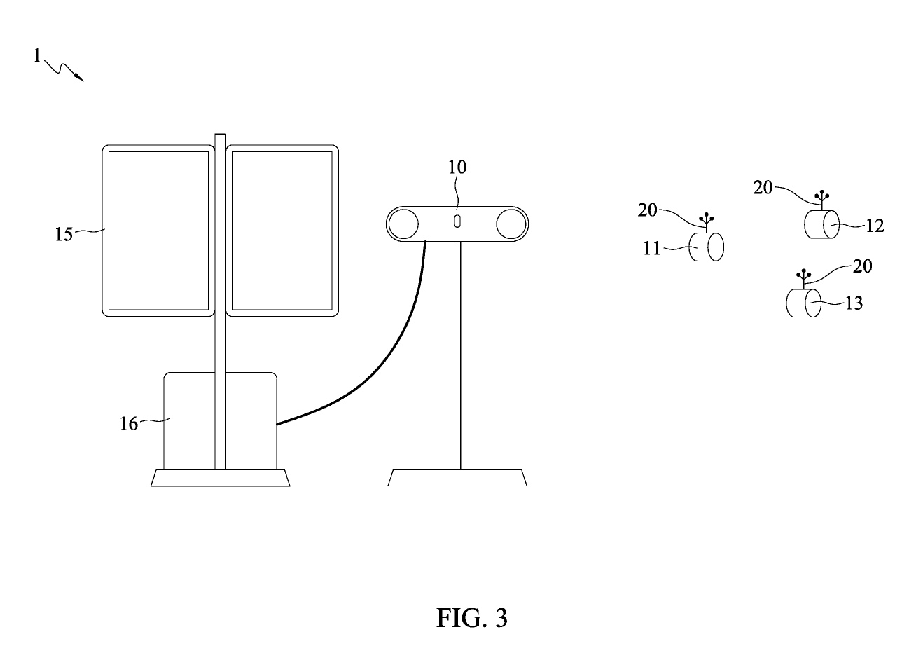

Referring to FIG. 3, a surgical navigation system 1 in accordance with the third embodiment of the present disclosure also includes a navigation unit 10, a first image-type projecting unit 11, a second image-type projecting unit 12, at least one third image-type projecting unit 13 and a processing unit 16. Only the differences between the third embodiment and the first embodiment are described below, while similar or the same technical contexts are omitted for conciseness.

The first image-type projecting unit 11, the second image-type projecting unit 12 and the third image-type projecting unit 13 are not provided on the navigation unit 10. The first, second and third image-type projecting units 11, 12 and 13 are stand-alone structures, such that it is convenient for surgeons to place the first, second and third image-type projecting units 11, 12 and 13 based on the surgery environment on site. During use, the relative positions of the first, second and third image-type projecting units 11, 12 and 13 and the navigation unit 10 are first calculated before projections can be made by the first, second and third image-type projecting units 11, 12 and 13. In other words, the relationships of the coordinate systems between the first image-type projecting unit 11, the second image-type projecting unit 12, the third image-type projecting unit 13 and the navigation unit 10 are not fixed. The locations of the image-type projecting units have to be located (i.e., via tracking balls 20) before coordinate conversions can be performed. In other words, R and t are not fixed and are determined through detection of the locations of the image-type projecting units in a real-time manner. In an embodiment, the present disclosure does not limit the number of image-type projecting units. Since the methods for locating the image-type projecting units have been described in the above embodiments, they will not be described again.

The above descriptions are related to implementations of the navigation unit 10 equipped with an infrared tracker. The embodiments below describe implementations pertaining to the use of ultrasound, CT, MRI or optical coherence tomography (OCT) in the navigation unit 10.

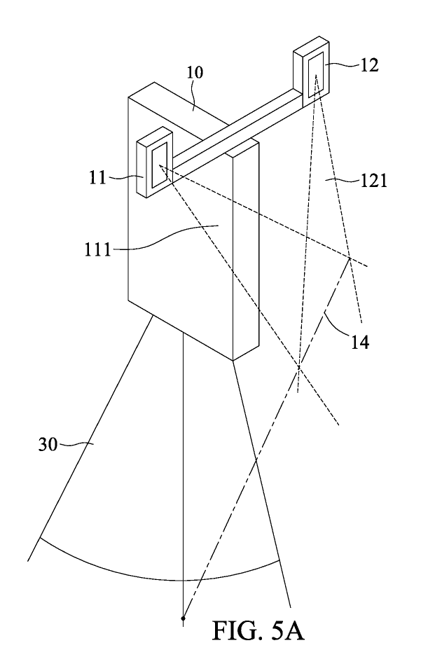

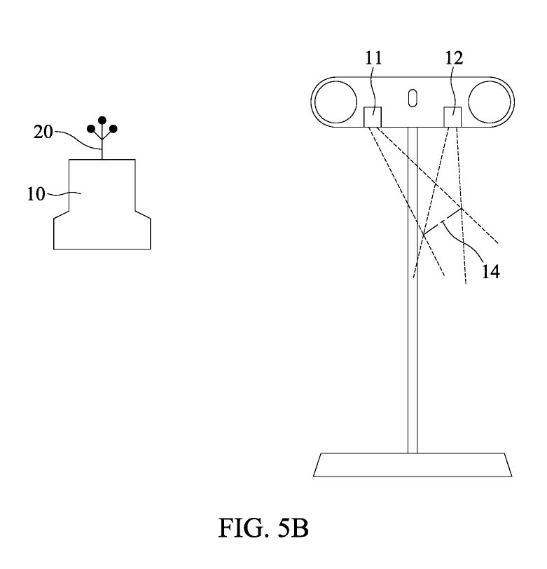

Referring to FIGS. 5A and 5B, a surgical navigation system 1 in accordance with the fourth embodiment and the fifth embodiment of the present disclosure also includes a navigation unit 10, a first image-type projecting unit 11, a second image-type projecting unit 12 and a processing unit (not shown). The technical aspects of the first and second image-type projecting units 11 and 12 of these embodiments have already been described before, and will not be repeated. Only the differences of the navigation unit 10 between this embodiment and the previous embodiments are described below.

As shown in FIG. 5A, the navigation unit 10 is an ultrasound probe provided with the first and second image-type projecting units 11 and 12. For simplicity, elements such as the processing unit and the display unit are not shown in FIG. 5A. However, those with ordinary skill in the art can appreciate how the processing unit and the display unit work in this embodiment based on the above descriptions. In this embodiment, images are obtained by ultrasound, such that the surgeon can decide the location and the angle of an entry point in real time during scanning of an image 30 of the patient's body during the operation. For example, a software user interface provided by the surgical navigation system 1 allows the surgeon to plan treatment for patients, such that the first and second image-type projecting units 11 and 12 project at least two patterns forming an intersection area 14 based on the decided location and angle of the entry point.

As shown in FIG. 5B, another implementation is shown, in which tracking balls 20 are equipped on the navigation unit 10 (i.e., the ultrasound probe), and an infrared tracker is provided on the first and second image-type projecting units 11 and 12 in order to establish the coordinate conversion relationships between the navigation unit 10 and the image-type projecting units. In an embodiment, the first and second image-type projecting units 11 and 12 can be stand-alone structures (e.g., such as those shown in FIG. 3) or provided on a supporting element (e.g., those shown in FIGS. 2 and 5B), but the present disclosure is not so limited. Similarly, other relevant elements such as the processing unit and the display unit are not shown in this diagram. However, those with ordinary skill in the art can appreciate how the processing unit and the display unit work in this embodiment based on the above descriptions.

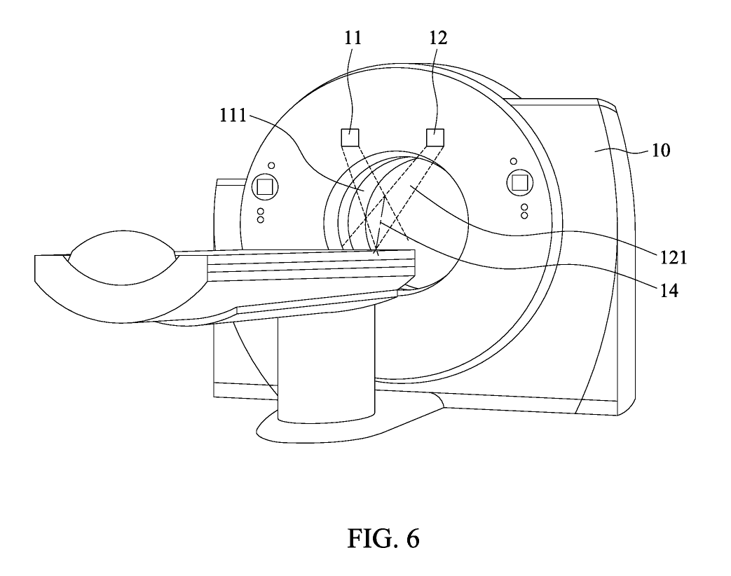

Referring to FIG. 6, a surgical navigation system 1 in accordance with the sixth embodiment of the present disclosure also includes a navigation unit 10, a first image-type projecting unit 11, a second image-type projecting unit 12 and a processing unit (not shown). The technical aspects of the first and second image-type projecting units 11 and 12 of these embodiments have already been described before, and will not be repeated. Only the differences of the navigation unit 10 between this embodiment and the previous embodiments are described below.

As shown in FIG. 6, the navigation unit 10 is a CT scanning equipment. The first and second image-type projecting units 11 and 12 are provided on the CT scanning equipment. After the CT scanning equipment has scanned the patient, the surgeon can directly plan the angle and the entry point on the screen of the display unit for surgery operation (that is, by using the software described before, for example). As the patient stays in the same place after the CT scan is taken, there is no need for registration. The first and second image-type projecting units 11 and 12 can then project at least two patterns forming an intersection area 14 based on the planned path for the entry point.

Similarly, in the case that the navigation unit 10 employs ultrasound or CT scan, the coordinate conversion relationships between the navigation unit 10 and the image-type projecting units can be fixed or not fixed. For the sake of convenience, the embodiments aforementioned only illustrate fixed relationships (e.g., the sixth embodiment illustrates the coordinate conversion relationships between the navigation unit 10 (i.e., CT equipment) and the image-type projecting units are fixed). Various implementation details are omitted as one with ordinary skill in the art can understand them based on the descriptions of the first embodiment to the third embodiment. It can be appreciated that in the case that the navigation unit 10 employs ultrasound or CT scan, if the coordinate conversion relationships between the navigation unit 10 and the image-type projecting units are not fixed, an additional location device (e.g., an optical or electromagnetic tracker) can be provided on the ultrasound/CT scanning equipment, while location sensing devices (e.g., a tracking balls) are provided on the image-type projecting units to locate the image-type projecting units.

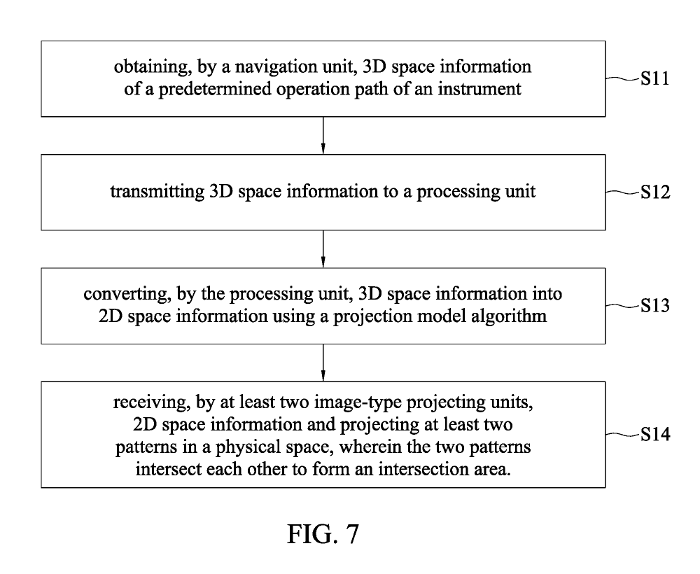

Referring to FIG. 7, an instrument guiding method for a surgical navigation system in accordance with an embodiment of the present disclosure is shown. The method includes steps S11-S14. In step S11, 3D space information of a predetermined operation path of an instrument is first obtained, wherein the 3D space information of the predetermined operation path of the instrument is obtained by a navigation unit using a tracking device, ultrasound, CT, MRI or OCT. Then, the method proceeds to step S12.

In step S12, the 3D space information is transmitted to a processing unit. In step S13, the 3D space information is converted into 2D space information using a projection model algorithm by the processing unit.

In an embodiment, the projection model algorithm is a perspective projection model, and has a formula as s\tilde{m}=P\tilde{M}, P=K[R|t], wherein M is the 3D space information of the instrument operation path under the coordinate system of the navigation unit, m is the 2D space information of the instrument operation path under the coordinate system of the projection, s is a scaling parameter, and P is a projection matrix that includes K being a projection calibration matrix, R being a rotational matrix and t being a translation vector. Then, the method proceeds to step S14.

In step S14, the 2D space information is received by at least two image-type projecting units, such that the image-type projecting units project at least two patterns in a physical space, wherein the two patterns intersect each other to form an intersection area, wherein the intersection area are in straight lines or curved lines.

In an embodiment, the relationships of the coordinate systems between the various image-type projecting units and the navigation unit are not fixed. In another embodiment, the relationships of the coordinate systems between the various image-type projecting units are fixed, while the relationships of the coordinate systems between the various image-type projecting units and the navigation unit are not fixed. In yet another embodiment, the relationships of the coordinate systems between the various image-type projecting units and the navigation unit are fixed.

In another embodiment of the present disclosure, a medium spreading unit can be used to spread a medium into the physical space to show the intersection area. In an embodiment, the medium can be a material with scattering characteristics (e.g., high-concentration Silicon dioxide, titanium dioxide, dry ice or other sterilized materials with high scattering coefficients).

With the surgical navigation system and the instrument guiding method for the same according to the present disclosure, the 3D space information of a predetermined operation path of an instrument can be converted into 2D space information using a processing unit, such that at least two image-type projecting units can project at least two patterns in the physical space. The intersection area of the patterns is the guiding path of the surgical instrument. As such, a surgeon does not need to switch focus between an image screen provided by a traditional surgical navigation system and the physical space of the patient where operation is being performed. The surgeon is able to start operation based on the guiding path of the surgical instrument. This increases convenience in operations of the surgery. In addition, as the surgical navigation system according to the present disclosure employs pico projectors, the components used can be miniaturized. Moreover, the image-type projecting units according to the present disclosure can form a project image plane, which is more advantageous than just dots and lines projected by the existing projectors in the prior art. The advantages include more complex pattern and versatile color image for guiding purpose. In other words, if Digital Light Processing (DLP) projecting devices or Liquid Crystal on Silicon (LCoS) projecting devices are used as the image-type projecting units according to the present disclosure, a projection plane can be created, whereas if Laser Beam Scanning (LBS) projecting devices are used, through MEMS fast scanning (raster scanning), a projection plane can be formed during the period of visual persistence. Furthermore, with the surgical navigation system according to the present disclosure and the instrument guiding method thereof, additional design for surgical tool with tracing markers attached is not necessary, reducing impact of sterilizing concerns during the design.

The above embodiments are only used to illustrate the principles of the present disclosure, and should not be construed as to limit the present disclosure in any way. The above embodiments can be modified by those with ordinary skill in the art without departing from the scope of the present disclosure as defined in the following appended claims.

What is claimed is:

1. A surgical navigation system, comprising:
a navigation unit configured for obtaining three-dimensional (3D) space information of a predetermined operation path of an instrument;
a processing unit configured for receiving the 3D space information and converting the 3D space information into two-dimensional (2D) space information using a projection model algorithm; and
at least two image-type projecting units configured for receiving the 2D space information respectively and projecting at least two patterns in a physical space, wherein the at least two patterns intersect each other to form an intersection area.

2. The surgical navigation system of claim 1, wherein the image-type projecting units are digital light processing (DLP) projecting devices, laser beam scanning (LBS) projecting devices, liquid crystal on silicon (LCoS) projecting devices, or any combination thereof.

3. The surgical navigation system of claim 1, wherein a relationship of coordinate systems between the image-type projecting units and the navigation unit is not fixed.

4. The surgical navigation system of claim 3, wherein a relationship of coordinate systems between the image-type projecting units is fixed.

5. The surgical navigation system of claim 1, wherein a relationship of coordinate systems between the image-type projecting units and the navigation unit is fixed.

6. The surgical navigation system of claim 1, wherein the navigation unit obtains the 3D space information of the predetermined operation path of the instrument by using a tracking device, ultrasound, computed tomography (CT), magnetic resonance imaging (MRI) or optical coherence tomography (OCT).

7. The surgical navigation system of claim 6, wherein the tracking device is an optical tracking device, an electromagnetic tracking device or a mechanical tracking device.

8. The surgical navigation system of claim 1, wherein the intersection area is a straight line or a curved line.

9. The surgical navigation system of claim 1, further comprising a medium spreading unit configured for spreading a medium into the physical space to show the intersection area, wherein the medium is a material with scattering characteristic.

10. An instrument guiding method for a surgical navigation system, comprising:
obtaining, by a navigation unit, three-dimensional (3D) space information of a predetermined operation path of an instrument;
transmitting the 3D space information to a processing unit and converting, by the processing unit, the 3D space information into two-dimensional (2D) space information using a projection model algorithm; and
receiving, by at least two image-type projecting units, the 2D space information and projecting at least two patterns in a physical space, wherein the two patterns intersect each other to form an intersection area.

11. The instrument guiding method of claim 10, wherein the image-type projecting units are digital light processing (DLP) projecting devices, laser beam scanning (LBS) projecting devices, liquid crystal on silicon (LCoS) projecting devices, or any combination thereof.

12. The instrument guiding method of claim 10, wherein the 3D space information of the predetermined operation path of the instrument is obtained by the navigation unit by using a tracking device, ultrasound, computed tomography (CT), magnetic resonance imaging (MRI) or optical coherence tomography (OCT).

13. The instrument guiding method of claim 12, wherein the tracking device is an optical tracking device, an electromagnetic tracking device or a mechanical tracking device.

14. The instrument guiding method of claim 10, wherein a relationship of coordinate systems between the image-type projecting units and the navigation unit is not fixed.

15. The instrument guiding method of claim 14, wherein a relationship of coordinate systems between the image-type projecting units is fixed.

16. The instrument guiding method of claim 10, wherein a relationship of coordinate systems between the image-type projecting units and the navigation unit is fixed.

17. The instrument guiding method of claim 10, wherein the intersection area is a straight line or a curved line.

18. The instrument guiding method of claim 10, further comprising spreading, by a medium spreading unit, a medium into the physical space to show the intersection area, wherein the medium is a material with scattering characteristic.
